(19) United States
(12) Patent Application Publication
(10) Pub. No.: US 2022/0409519 A1
(43) Pub. Date: Dec. 29, 2022

(54) COSMETIC USE OF AN AROMATIC RAW MATERIAL EXTRACT FOR PROTECTING SKIN FROM BLUE LIGHT

(71) Applicant: ROBERTET S.A., Grasse (FR)

(72) Inventor: Anthony PEGARD, Grasse (FR)

(73) Assignee: ROBERTET S.A., Grasse (FR)

(21) Appl. No.: 17/773,765

(22) PCT Filed: Nov. 12, 2020
(86) PCT No.: PCT/FR2020/052065

§ 371 Date: May 2, 2022

(30) Foreign Application Priority Data
Nov. 13, 2019 (FR) 1912656

(51) Int. Cl. A61K 8/9789; A61K 8/9794; A61Q 17/04; A61K 8/37

(52) U.S. Cl. CPC A61K 8/9789; A61K 8/9794; A61Q 17/04; A61K 8/375

(57) ABSTRACT

Methods for protecting the skin from blue light comprising administering to a subject in need thereof at least one extract of aromatic raw material selected from the group consisting of a plant extract of rosemary containing at least 1% of rosmarinic acid, a black pepper absolute, a gentian absolute, a myrrh resinoid, an essential oil of mandarin petitgrain, a ginger absolute, and a tea resinoid.

The present invention relates to the cosmetic use of one or more aromatic raw material extract(s) selected from a tea resinoid, a plant extract of rosemary, a black pepper absolute, a gentian absolute, essential oil of mandarin petitgrain, ginger absolute and myrrh resinoid.

The blue light is a light in which wavelengths shorter than those of the illuminant dominate, that is to say that its proximal color temperature is higher than that of the main light. A light where the wavelengths of 380 to 450 nanometers strongly dominate, beyond the capacities of visual chromatic adaptation, will always be blue.

The sky, on a sunny day, is a source of blue light, but the expression «blue light» is generally used for artificial lights. It is the blue glow of the electric arc and the blue light of the Cherenkov effect.

The screens of televisions, computers, smartphones, digital tablets, etc., emit a high proportion of blue light.

The effects of blue light on the eye, more specifically on the retina, are now well known. However, the effects of blue light on our skin are much less so. Nevertheless, like the sun, pollution, tobacco or stress, blue light promotes skin aging through inflammation, leading to the premature appearance of fine lines and wrinkles. Indeed, blue light over-stimulates the melanocytes which, when they are overstressed, produce more melanin, which promotes the appearance of spots.

Although the use of «conventional» sun creams is sometimes considered for protecting the skin from blue light, it remains preferable to use compositions specifically dedicated to this type of protection, which are generally more effective.

The absolute is an extract, generally obtained from plant material, widely used in the perfume industry. Unlike the plant extracts traditionally used in cosmetics, the absolute is obtained by alcoholic transformation of a concrete itself obtained by extraction with a volatile organic solvent.

More specifically, the absolute is obtained from concrete, which is generally prepared by maceration of a fresh plant (flowers, roots, leaves, bark, berries, etc.) in a hydrocarbon-based volatile organic solvent such as hexane or petroleum ether, or a bi-solvent mixture such as hexane/ethyl acetate or hexane/isopropyl acetate or hexane/isopropyl alcohol; in supercritical CO₂; or in a liquefied gas (or a mixture of liquefied gases) such as butane or HFC134a. The solvent is then evaporated to give a paste called concrete which contains aromatic compounds, waxes and oily compounds of the plant.

The concrete, which contains waxes responsible for the turbidity of the solution and make it poorly soluble in the base of the perfume, cannot be used in alcoholic perfumery as such but can be used in soaps.

The elimination, at least partial, of the waxes is therefore necessary. This elimination is obtained by glazing and filtration: after adding alcohol to the concrete, the alcoholic solution obtained is homogenized with vigorous stirring at a temperature of approximately 30° C. to 60° C. then refrigerated between −5° C. and −18° C. so that waxes can be eliminated by precipitation and filtration from concrete. The alcohol is then evaporated to obtain the absolute.

The resinoid is an extract of a dry raw material, generally a vegetable material, obtained by extraction using a non-aqueous solvent. The resinoid is obtained by alcoholic transformation of a concrete itself obtained by extraction with a polar volatile solvent. More precisely, the resinoid is obtained from concrete, which is generally prepared by macerating a dry plant in a polar volatile organic solvent such as ethanol, or a mixture of polar solvents such as ethanol/acetone. The solvent is then evaporated to give a paste called concrete which contains aromatic compounds, and oily compounds of the plant.

The concrete cannot be used in alcoholic perfumery as such but can be used in soaps.

The elimination, at least partial, of fats is therefore necessary. This elimination is obtained by glazing and filtration: after adding hot alcohol to the concrete, the alcoholic solution obtained is homogenized with vigorous stirring at a temperature of approximately 30° C. to 60° C. then refrigerated between −5° C. and −18° C. so that fatty substances can be eliminated by precipitation and filtration. The alcohol is then evaporated to obtain the resinoid.

Thus, the use of absolutes or resinoids in perfumery is known.

Chinese patent application CN105919904 describes a cosmetic composition comprising in particular 3 to 7% of a marigold flower extract; 8 to 12% rosemary leaf oil; 3 to 7% cocoa seed extract; and 3 to 7% tremella extract to protect the skin from the effects of blue light. Rosemary leaf extract is obtained by supercritical CO2 extraction. As explained by O. Yesi et al. in In vitro antioxidant activities of Rosmarinus officinalis extracts treated with supercritical carbon dioxide, Food chemistry 101, 1474-1481, (2001), such an extraction does not make it possible to recover certain components, in particular rosmarinic acid.

The Chinese patent application CN109549893 describes a cosmetic composition comprising in particular a chlorella extract; a crambe extract; a ginger flower root extract; and a spring elm extract to protect the skin from the effects of blue light. The used ginger extract is rich in carbohydrates which indicates that it is not an absolute but an aqueous extract.

Finally, the Chinese patent application CN109464308 describes a cosmetic composition comprising in particular from 0.05 to 10% essential oil of nectar; from 0.05 to 6% pomegranate essential oil; 0.1 to 5% ginger essential oil; and 0.1 to 10% lavender essential oil; to protect the skin from the effects of blue light. This composition comprises ginger essential oil but does not contain its absolute.

Yet, it has now surprisingly been found that other extracts (such as resinoids, absolutes or plant extracts) of aromatic raw materials can effectively protect the skin against the effects of blue light.

Thus, the present invention relates to the cosmetic use of one or more extract(s) of aromatic raw material selected from a plant extract of rosemary containing at least 1% of rosmarinic acid, a black pepper absolute, gentian absolute, myrrh resinoid, essential oil of mandarin petitgrain, ginger absolute and tea resinoid to protect the skin from blue light.

In the context of the present invention, the term «absolute» means any extract of plant material capable of being obtained by any method known to one skilled in the art, in particular the following method:
preparation of a concrete in the form of a paste
   by maceration of fresh plant material in a volatile organic hydrocarbon solvent such as hexane or petroleum ether, or a mixture of solvents such as hexane/ethyl acetate or hexane/isopropyl acetate or hexane/alcohol isopropyl; in supercritical CO₂; or in a liquefied gas (or a mixture of liquefied gases) such as butane or HFC134a;
   followed by the evaporation of the solvent or solvent mixture;
elimination, at least partial, of the waxes contained in the concrete thus obtained to obtain the absolute by
   addition of alcohol;
   homogenization of the solution thus obtained under vigorous stirring at a temperature of approximately 30° C. to 60° C.;
   refrigeration of the solution between −5° C. and −18° C. and elimination of waxes from concrete by precipitation and filtration;
   and evaporation of alcohol.

The method for preparing the absolute can be followed, if necessary, by a step for purifying the absolute by any technique known to one skilled in the art, in particular by molecular distillations or by using ion exchange resins.

Absolutes can be obtained from all parts of the considered plant, such as for example the bud, the cone, the needles, the flower, the root, the sap, the bark, the berry, the stem or the leaf.

In the context of the present invention, the term «resinoid» means any extract of plant material capable of being obtained by any method known to one skilled in the art, in particular the following method:
preparation of a concrete in the form of a paste
   by maceration of the dry plant material in a polar volatile organic solvent such as ethanol or acetone, or a mixture of solvents such as ethanol/acetone.
   followed by evaporation of the solvent or solvent mixture;
elimination, at least partial, of the oily substances contained in the concrete thus obtained to obtain the resinoid by
   addition of alcohol;
   homogenization of the solution thus obtained under vigorous stirring at a temperature of approximately 30° C. to 60° C.;
   refrigeration of the solution between −5° C. and −18° C. and elimination of fatty substances from concrete, by precipitation and filtration;
   and evaporation of alcohol.

The method for preparing resinoid can be followed, if necessary, by a purification step by any technique known to one skilled in the art, in particular by molecular distillations or by using ion exchange resins.

The resinoids can be obtained from the dry parts of the considered plant, such as for example the root, the sap, the bark, the dried leaf.

In the context of the present invention, the term «plant extract» means any plant extract obtained by extraction using a polar solvent such as ethyl acetate.

In addition, in the context of the present invention:
the term «rosemary» means any species of rosemary, and in particular the Rosmarinus officinalis species;
the term «black pepper» means any species of black pepper, and in particular the Piper nigra species;
the term «gentian» means any species of gentian, and in particular the Gentiana lutea species;
the term «myrrh» means any species of myrrh, and in particular the Commiphora myrrha species;
the term «essential oil of mandarin petitgrain» means any oil obtained by steam distillation of the leaves, branches and green fruits of the mandarin tree, and in particular of the Citrus reticulata species;
the term «ginger» means any species of ginger, and in particular the Zingiber officinale species;
the term «tea» means any species of tea, and in particular the Camelia sinensis species; and
the term «cosmetic use for protecting the skin from blue light» means any non-therapeutic use of a product with a view to limit the harmful effects of blue light on the skin, in particular the acceleration of skin aging and the loss of elasticity of the skin.

Finally, in the context of the present invention, and unless otherwise stated, the proportions expressed in % correspond to mass percentages relative to the total weight of the considered entity.

The present invention therefore relates to the cosmetic use of one or more extract(s) for protecting the skin from blue light. Preferably, said extract(s) have all or part of the following characteristics:
the plant extract of rosemary is obtained from spent grains of rosemary needles, more preferably from spent grains of Rosmarinus officinalis needles;
the plant extract of rosemary contains at least 5% rosmarinic acid, more preferably at least 10% rosmarinic acid, preferably at least 20% rosmarinic acid, most preferably at least 30% rosmarinic acid;
the absolute obtained from black pepper is obtained from black pepper berries; more preferably from Piper nigra berries;
the absolute obtained from gentian is obtained from gentian roots, more preferably from Gentiana lutea roots;
the resinoid obtained from myrrh is obtained from myrrh resin, more preferably from Commiphora myrrha resin;
the absolute obtained from ginger is obtained from ginger rhizomes, more preferably from Zingiber officinale rhizomes;
the absolute obtained from ginger contains more than 40% dry matter, more preferably more than 50% dry matter, more preferably more than 60% dry matter, most preferably more than 70% dry matter; and or
the resinoid obtained from tea is obtained from tea leaves, more preferably from Camelia sinensis leaves.

In the context of the present invention, the extracts cited above can be used alone or in combination. Thus, the present invention preferably relates to the cosmetic use of one of the following combinations of extracts to protect the skin from blue light:
a plant extract of rosemary as defined above and a black pepper absolute as defined above;
a plant extract of rosemary as defined above and a gentian absolute as defined above;
a plant extract of rosemary as defined above and a myrrh resinoid as defined above;
a plant extract of rosemary as defined above and an essential oil of mandarin petitgrain as defined above;
a plant extract of rosemary as defined above and a ginger absolute as defined above;
a plant extract of rosemary as defined above and a tea resinoid as defined above;
a black pepper absolute as defined above and a gentian absolute as defined above;
a black pepper absolute as defined above and a myrrh resinoid as defined above;
a black pepper absolute as defined above and an essential oil of mandarin petitgrain as defined above;
a black pepper absolute as defined above and a ginger absolute as defined above;
a black pepper absolute as defined above and a tea resinoid as defined above;
a gentian absolute as defined above and a myrrh resinoid as defined above;
a gentian absolute as defined above and an essential oil of mandarin petitgrain as defined above;
a gentian absolute as defined above and a ginger absolute as defined above;
a gentian absolute as defined above and a tea resinoid as defined above;
a myrrh resinoid as defined above and an essential oil of mandarin petitgrain as defined above;
a myrrh resinoid as defined above and a ginger absolute as defined above;
a myrrh resinoid as defined above and a tea resinoid as defined above;
an essential oil of mandarin petitgrain as defined above and a ginger absolute as defined above;
an essential oil of mandarin petitgrain as defined above and a tea resinoid as defined above;
a ginger absolute as defined above and a tea resinoid as defined above;
a plant extract of rosemary as defined above, a black pepper absolute as defined above and a gentian absolute as defined above;
a plant extract of rosemary as defined above, a black pepper absolute as defined above and a myrrh resinoid as defined above;
a plant extract of rosemary as defined above, a black pepper absolute as defined above and an essential oil of mandarin petitgrain as defined above;
a plant extract of rosemary as defined above, a black pepper absolute as defined above and a ginger absolute as defined above;
a plant extract of rosemary as defined above, a black pepper absolute as defined above and a tea resinoid as defined above;
a plant extract of rosemary as defined above, a gentian absolute as defined above and a myrrh resinoid as defined above;
a plant extract of rosemary as defined above, a gentian absolute as defined above and an essential oil of mandarin petitgrain as defined above;
a plant extract of rosemary as defined above, a gentian absolute as defined above and a ginger absolute as defined above;
a plant extract of rosemary as defined above, a gentian absolute as defined above and a tea resinoid as defined above;
a plant extract of rosemary as defined above, a myrrh resinoid as defined above and an essential oil of mandarin petitgrain as defined above;
a plant extract of rosemary as defined above, a myrrh resinoid as defined above and a ginger absolute as defined above;
a plant extract of rosemary as defined above, a myrrh resinoid as defined above and a tea resinoid as defined above;
a plant extract of rosemary as defined above, an essential oil of mandarin petitgrain as defined above and a ginger absolute as defined above;
a plant extract of rosemary as defined above, an essential oil of mandarin petitgrain as defined above and a tea resinoid as defined above;
a plant extract of rosemary as defined above, a ginger absolute as defined above and a tea resinoid as defined above;
a black pepper absolute as defined above, a gentian absolute as defined above and a myrrh resinoid as defined above;
a black pepper absolute as defined above, a gentian absolute as defined above and an essential oil of mandarin petitgrain as defined above;
a black pepper absolute as defined above, a gentian absolute as defined above and a ginger absolute as defined above;
a black pepper absolute as defined above, a gentian absolute as defined above and a tea resinoid as defined above;
a black pepper absolute as defined above, a myrrh resinoid as defined above and an essential oil of mandarin petitgrain as defined above;
a black pepper absolute as defined above, a myrrh resinoid as defined above and a ginger absolute as defined above;
a black pepper absolute as defined above, a myrrh resinoid as defined above and a tea resinoid as defined above;
a black pepper absolute as defined above, an essential oil of mandarin petitgrain as defined above and a ginger absolute as defined above;
a black pepper absolute as defined above, an essential oil of mandarin petitgrain as defined above and a tea resinoid as defined above;
a black pepper absolute as defined above, a ginger absolute as defined above and a tea resinoid as defined above;
a gentian absolute as defined above, a myrrh resinoid as defined above and an essential oil of mandarin petitgrain as defined above;
a gentian absolute as defined above, a myrrh resinoid as defined above and a ginger absolute as defined above;
a gentian absolute as defined above, a myrrh resinoid as defined above and a tea resinoid as defined above;
a gentian absolute as defined above, an essential oil of mandarin petitgrain as defined above and a ginger absolute as defined above;
a gentian absolute as defined above, an essential oil of mandarin petitgrain as defined above and a tea resinoid as defined above;
a gentian absolute as defined above, a ginger absolute as defined above and a tea resinoid as defined above;
a myrrh resinoid as defined above, an essential oil of mandarin petitgrain as defined above and a ginger absolute as defined above;
a myrrh resinoid as defined above, an essential oil of mandarin petitgrain as defined above and a tea resinoid as defined above;
a myrrh resinoid as defined above, a ginger absolute as defined above and a tea resinoid as defined above;
an essential oil of mandarin petitgrain as defined above, a ginger absolute as defined above and a tea resinoid as defined above;
a plant extract of rosemary as defined above, a black pepper absolute as defined above, a gentian absolute as defined above and a myrrh resinoid as defined above;
a plant extract of rosemary as defined above, a black pepper absolute as defined above, a gentian absolute as defined above and an essential oil of mandarin petitgrain as defined above;
a plant extract of rosemary as defined above, a black pepper absolute as defined above, a gentian absolute as defined above and a ginger absolute as defined above;
a plant extract of rosemary as defined above, a black pepper absolute as defined above, a gentian absolute as defined above and a tea resinoid as defined above;
a plant extract of rosemary as defined above, a black pepper absolute as defined above, a myrrh resinoid as defined above and an essential oil of mandarin petitgrain as defined above;
a plant extract of rosemary as defined above, a black pepper absolute as defined above, a myrrh resinoid as defined above and a ginger absolute as defined above;
a plant extract of rosemary as defined above, a black pepper absolute as defined above, a myrrh resinoid as defined above and a tea resinoid as defined above;
a plant extract of rosemary as defined above, a black pepper absolute as defined above, an essential oil of mandarin petitgrain as defined above and a ginger absolute as defined above;
a plant extract of rosemary as defined above, a black pepper absolute as defined above, an essential oil of mandarin petitgrain as defined above and a tea resinoid as defined above;
a plant extract of rosemary as defined above, a black pepper absolute as defined above, a ginger absolute as defined above and a tea resinoid as defined above;
a plant extract of rosemary as defined above, a gentian absolute as defined above, a myrrh resinoid as defined above and an essential oil of mandarin petitgrain as defined above;
a plant extract of rosemary as defined above, a gentian absolute as defined above, a myrrh resinoid as defined above and a ginger absolute as defined above;
a plant extract of rosemary as defined above, a gentian absolute as defined above, a myrrh resinoid as defined above and a tea resinoid as defined above;
a plant extract of rosemary as defined above, a gentian absolute as defined above, an essential oil of mandarin petitgrain as defined above and a ginger absolute as defined above;
a plant extract of rosemary as defined above, a gentian absolute as defined above, an essential oil of mandarin petitgrain as defined above and a tea resinoid as defined above;
a plant extract of rosemary as defined above, a gentian absolute as defined above, a ginger absolute as defined above and a tea resinoid as defined above;
a plant extract of rosemary as defined above, a myrrh resinoid as defined above, an essential oil of mandarin petitgrain as defined above and a ginger absolute as defined above;
a plant extract of rosemary as defined above, a myrrh resinoid as defined above, an essential oil of mandarin petitgrain as defined above and a tea resinoid as defined above;
a plant extract of rosemary as defined above, a myrrh resinoid as defined above, a ginger absolute as defined above and a tea resinoid as defined above;
a plant extract of rosemary as defined above, an essential oil of mandarin petitgrain as defined above, a ginger absolute as defined above and a tea resinoid as defined above;
a black pepper absolute as defined above, a gentian absolute as defined above, a myrrh resinoid as defined above and an essential oil of mandarin petitgrain as defined above;
a black pepper absolute as defined above, a gentian absolute as defined above, a myrrh resinoid as defined above and a ginger absolute as defined above;
a black pepper absolute as defined above, a gentian absolute as defined above, a myrrh resinoid as defined above and a tea resinoid as defined above;
a black pepper absolute as defined above, a gentian absolute as defined above, an essential oil of mandarin petitgrain as defined above and a ginger absolute as defined above;
a black pepper absolute as defined above, a gentian absolute as defined above, an essential oil of mandarin petitgrain as defined above and a tea resinoid as defined above;
a black pepper absolute as defined above, a gentian absolute as defined above, a ginger absolute as defined above and a tea resinoid as defined above;
a black pepper absolute as defined above, a myrrh resinoid as defined above, an essential oil of mandarin petitgrain as defined above and a ginger absolute as defined above;
a black pepper absolute as defined above, a myrrh resinoid as defined above, an essential oil of mandarin petitgrain as defined above and a tea resinoid as defined above;
a black pepper absolute as defined above, a myrrh resinoid as defined above, a ginger absolute as defined above and a tea resinoid as defined above;
a black pepper absolute as defined above, an essential oil of mandarin petitgrain as defined above, a ginger absolute as defined above and a tea resinoid as defined above;
a gentian absolute as defined above, a myrrh resinoid as defined above, an essential oil of mandarin petitgrain as defined above and a ginger absolute as defined above;
a gentian absolute as defined above, a myrrh resinoid as defined above, an essential oil of mandarin petitgrain as defined above and a tea resinoid as defined above;
a gentian absolute as defined above, a myrrh resinoid as defined above, a ginger absolute as defined above and a tea resinoid as defined above;
a gentian absolute as defined above, an essential oil of mandarin petitgrain as defined above, a ginger absolute as defined above and a tea resinoid as defined above;
a myrrh resinoid as defined above, an essential oil of mandarin petitgrain as defined above, a ginger absolute as defined above and a tea resinoid as defined above;
a plant extract of rosemary as defined above, a black pepper absolute as defined above, a gentian absolute as defined above, a myrrh resinoid as defined above and an essential oil of mandarin petitgrain as defined above;
a plant extract of rosemary as defined above, a black pepper absolute as defined above, a gentian absolute as defined above, a myrrh resinoid as defined above and a ginger absolute as defined above;
a plant extract of rosemary as defined above, a black pepper absolute as defined above, a gentian absolute as defined above, a myrrh resinoid as defined above and a tea resinoid as defined above;
a plant extract of rosemary as defined above, a black pepper absolute as defined above, a gentian absolute as defined above, an essential oil of mandarin petitgrain as defined above and a ginger absolute as defined above;
a plant extract of rosemary as defined above, a black pepper absolute as defined above, a gentian absolute as defined above, an essential oil of mandarin petitgrain as defined above and a tea resinoid as defined above;
a plant extract of rosemary as defined above, a black pepper absolute as defined above, a gentian absolute as defined above, a ginger absolute as defined above and a tea resinoid as defined above;
a plant extract of rosemary as defined above, a black pepper absolute as defined above, a myrrh resinoid as defined above, an essential oil of mandarin petitgrain as defined above and a ginger absolute as defined above;
a plant extract of rosemary as defined above, a black pepper absolute as defined above, a myrrh resinoid as defined above, an essential oil of mandarin petitgrain as defined above and a tea resinoid as defined above;
a plant extract of rosemary as defined above, a black pepper absolute as defined above, a myrrh resinoid as defined above, a ginger absolute as defined above and a tea resinoid as defined above;
a plant extract of rosemary as defined above, a black pepper absolute as defined above, an essential oil of mandarin petitgrain as defined above, a ginger absolute as defined above and a tea resinoid as defined above;
a plant extract of rosemary as defined above, a gentian absolute as defined above, a myrrh resinoid as defined above, an essential oil of mandarin petitgrain as defined above and a ginger absolute as defined above;
a plant extract of rosemary as defined above, a gentian absolute as defined above, a myrrh resinoid as defined above, an essential oil of mandarin petitgrain as defined above and a tea resinoid as defined above;
a plant extract of rosemary as defined above, a gentian absolute as defined above, a myrrh resinoid as defined above, a ginger absolute as defined above and a tea resinoid as defined above;
a plant extract of rosemary as defined above, a gentian absolute as defined above, an essential oil of mandarin petitgrain as defined above, a ginger absolute as defined above and a tea resinoid as defined above;
a plant extract of rosemary as defined above, a myrrh resinoid as defined above, an essential oil of mandarin petitgrain as defined above, a ginger absolute as defined above and a tea resinoid as defined above;
a black pepper absolute as defined above, a gentian absolute as defined above, a myrrh resinoid as defined above, an essential oil of mandarin petitgrain as defined above and a ginger absolute as defined above;
a black pepper absolute as defined above, a gentian absolute as defined above, a myrrh resinoid as defined above, an essential oil of mandarin petitgrain as defined above and a tea resinoid as defined above;
a black pepper absolute as defined above, a gentian absolute as defined above, a myrrh resinoid as defined above, a ginger absolute as defined above and a tea resinoid as defined above;
a black pepper absolute as defined above, a gentian absolute as defined above, an essential oil of mandarin petitgrain as defined above, a ginger absolute as defined above and a tea resinoid as defined above;
a black pepper absolute as defined above, a myrrh resinoid as defined above, an essential oil of mandarin petitgrain as defined above, a ginger absolute as defined above and a tea resinoid as defined above;
a gentian absolute as defined above, a myrrh resinoid as defined above, an essential oil of mandarin petitgrain as defined above, a ginger absolute as defined above and a tea resinoid as defined above;
a plant extract of rosemary as defined above, a black pepper absolute as defined above, a gentian absolute as defined above, a myrrh resinoid as defined above, an essential oil of mandarin petitgrain as defined above and a ginger absolute as defined above;
a plant extract of rosemary as defined above, a black pepper absolute as defined above, a gentian absolute as defined above, a myrrh resinoid as defined above, an essential oil of mandarin petitgrain as defined above and a tea resinoid as defined above;
a plant extract of rosemary as defined above, a black pepper absolute as defined above, a gentian absolute as defined above, a myrrh resinoid as defined above, a ginger absolute as defined above and a resinoid tea as defined above;
a plant extract of rosemary as defined above, a black pepper absolute as defined above, a gentian absolute as defined above, an essential oil of mandarin petitgrain as defined above, a ginger absolute as defined above and a tea resinoid as defined above;
a plant extract of rosemary as defined above, a black pepper absolute as defined above, a myrrh resinoid as defined above, an essential oil of mandarin petitgrain as defined above, a ginger absolute as defined above and a tea resinoid as defined above;
a plant extract of rosemary as defined above, a gentian absolute as defined above, a myrrh resinoid as defined above, an essential oil of mandarin petitgrain as defined above, a ginger absolute as defined above and a tea resinoid as defined above;
a black pepper absolute as defined above, a gentian absolute as defined above, a myrrh resinoid as defined above, an essential oil of mandarin petitgrain as defined above, a ginger absolute as defined above and a tea resinoid as defined above; or
a plant extract of rosemary as defined above, a black pepper absolute as defined above, a gentian absolute as defined above, a myrrh resinoid as defined above, an essential oil of mandarin petitgrain as defined above, a ginger absolute as defined above and a tea resinoid as defined above.

Preferably, the object of the present invention is the combined use of a plant extract of rosemary as defined above, of a black pepper absolute as defined above and of a gentian absolute as defined above to protect skin from blue light.

More preferably, the object of the present invention is the combined use of a plant extract of rosemary as defined above, of a black pepper absolute as defined above, of a gentian absolute as defined above and of a myrrh resinoid as defined above to protect the skin from blue light.

More preferably, the object of the present invention is the use of a plant extract of rosemary as defined above, of a black pepper absolute as defined above, of a gentian absolute as defined above, of a myrrh resinoid as defined above, an essential oil of mandarin petitgrain as defined above and a ginger absolute as defined above to protect the skin from blue light.

Quite preferably, the object of the present invention is the combined use of a plant extract of rosemary as defined above, of a black pepper absolute as defined above, of a gentian absolute as defined above, a myrrh resinoid as defined above, an essential oil of mandarin petitgrain as defined above, a ginger absolute as defined above and a resinoid obtained from tea as defined above to protect the skin from blue light.

The extracts described in the context of the present invention are therefore used in cosmetics to protect the skin from blue light. To do this, these extracts can be incorporated into a cosmetic composition which can be formulated in any pharmaceutical form suitable for its administration, in particular for its application to the skin (topically).

The cosmetic compositions comprising the extract(s) used in the context of the present invention can be formulated in liquid, pasty or solid form, and more particularly in the form of ointments, creams, milks, pomades, powders, soaked pads (wipes), solutions, gels, sprays, foams, suspensions or sticks. They can also be in the form of suspensions of microspheres or nanospheres, of lipid or polymeric vesicles, or of polymeric or gelled patches allowing controlled release, but also in the form of rinse-off products such as shower gels, bath products (salts, foaming, etc.).

These cosmetic compositions contain the extract(s) used in the context of the present invention at contents ranging from 0.0001% to 10% by total weight of the composition, preferably from 0.001% to 1% by total weight of the composition.

For the preparation of these compositions, the extract(s) used in the context of the present invention is (or are) mixed with the excipients conventionally used in the cosmetics field.

The compositions comprising the extract(s) used in the context of the present invention can take the form of perfuming compositions having a cosmetic activity, such as for example the compositions of the Actiscent® range.

The cosmetic compositions comprising the extract(s) used in the context of the present invention can take the form of a cream in which said fraction is combined with the excipients commonly used in cosmetology.

These compositions can also take the form of gels in the appropriate excipients such as cellulose esters or other gelling agents, such as carbopol, sepinov (polyacrylate) or guar gum.

These compositions can also take the form of a lotion or a solution in which the extract used in the context of the present invention is in encapsulated form. The microspheres can for example consist of fatty substances, agar and water. The extract used in the context of the present invention can be incorporated into vectors of the liposome, glycosphere, cyclodextrin type, into chylomicrons, macro-, micro-, nano-particles as well as macro-, micro- and nano-capsules and also be absorbed on powdery organic polymers, talcs, bentonites and other mineral supports.

These emulsions have good stability and can be stored for the time necessary for the use at temperatures comprised between 0 and 50° C. without there being any sedimentation of the constituents or separation of the phases.

The cosmetic compositions comprising the extract(s) used in the context of the present invention may also contain additives or adjuvants customary in cosmetology, such as for example antimicrobial agents or perfumes, but also extraction lipids or synthetic, gelling and viscosifying polymers, surfactants and emulsifiers, water- or fat-soluble active ingredients, plant extracts, tissue extracts, marine extracts, synthetic active ingredients.

The cosmetic compositions comprising the extract(s) used in the context of the present invention may also comprise other complementary active principles selected for their action, for example for the slimming effect, the anti-cellulite effect, firming effect, moisturizing effect, anti-aging effect, antimicrobial activity, antioxidant activity, anti-radical activity, healing effect, tightening effect, anti-wrinkle, chelating activity, complexing and sequestering activity, soothing effect, concealer effect, anti-redness effect, emollient activity, hair detangling effect, anti-dandruff activity, the stimulating effect of hair regrowth, the effect of inhibiting hair loss, the capillary sheathing effect, the depilatory activity, the activity limiting hair regrowth, the activity involved in cell renewal, the activity modulating the inflammatory response, the activity helping to maintain the oval of the face, but also sun protection, anti-irritant activity, cellular nutrition, cellular respiration, anti-seborrheic treatments, skin tone, hair protection. When the cosmetic compositions comprising the extract(s) used in the context of the present invention contain complementary active principles, these are generally present in the composition at a sufficiently high concentration for them to be able to exert their activity.

The cosmetic compositions comprising the extract(s) used in the context of the present invention are preferably daily used and applied one or more times per day. They can be applied to all areas requiring protection from blue light, in particular on the face.

Some of the cosmetic compositions described above are new. Thus, the present invention also relates to a cosmetic composition (for protecting the skin from blue light) comprising at least three extracts selected from a plant extract of rosemary as defined above, a black pepper absolute as defined above, a gentian absolute as defined above, a myrrh resinoid as defined above, an essential oil of mandarin petitgrain as defined above, a ginger absolute as defined above and a resinoid obtained from tea as defined above.

As examples of cosmetic compositions according to the present invention, mention may in particular be made of cosmetic compositions (for protecting the skin from blue light) comprising the following combinations of extracts:
a plant extract of rosemary as defined above, a black pepper absolute as defined above and a gentian absolute as defined above;
a plant extract of rosemary as defined above, a black pepper absolute as defined above and a myrrh resinoid as defined above;
a plant extract of rosemary as defined above, a black pepper absolute as defined above and an essential oil of mandarin petitgrain as defined above;
a plant extract of rosemary as defined above, a black pepper absolute as defined above and a ginger absolute as defined above;
a plant extract of rosemary as defined above, a black pepper absolute as defined above and a tea resinoid as defined above;
a plant extract of rosemary as defined above, a gentian absolute as defined above and a myrrh resinoid as defined above;
a plant extract of rosemary as defined above, a gentian absolute as defined above and an essential oil of mandarin petitgrain as defined above;
a plant extract of rosemary as defined above, a gentian absolute as defined above and a ginger absolute as defined above;
a plant extract of rosemary as defined above, a gentian absolute as defined above and a tea resinoid as defined above;
a plant extract of rosemary as defined above, a myrrh resinoid as defined above and an essential oil of mandarin petitgrain as defined above;
a plant extract of rosemary as defined above, a myrrh resinoid as defined above and a ginger absolute as defined above;
a plant extract of rosemary as defined above, a myrrh resinoid as defined above and a tea resinoid as defined above;
a plant extract of rosemary as defined above, an essential oil of mandarin petitgrain as defined above and a ginger absolute as defined above;
a plant extract of rosemary as defined above, an essential oil of mandarin petitgrain as defined above and a tea resinoid as defined above;
a plant extract of rosemary as defined above, a ginger absolute as defined above and a tea resinoid as defined above;
a black pepper absolute as defined above, a gentian absolute as defined above and a myrrh resinoid as defined above;
a black pepper absolute as defined above, a gentian absolute as defined above and an essential oil of mandarin petitgrain as defined above;
a black pepper absolute as defined above, a gentian absolute as defined above and a ginger absolute as defined above;
a black pepper absolute as defined above, a gentian absolute as defined above and a tea resinoid as defined above;
a black pepper absolute as defined above, a myrrh resinoid as defined above and an essential oil of mandarin petitgrain as defined above;
a black pepper absolute as defined above, a myrrh resinoid as defined above and a ginger absolute as defined above;
a black pepper absolute as defined above, a myrrh resinoid as defined above and a tea resinoid as defined above;
a black pepper absolute as defined above, an essential oil of mandarin petitgrain as defined above and a ginger absolute as defined above;
a black pepper absolute as defined above, an essential oil of mandarin petitgrain as defined above and a tea resinoid as defined above;
a black pepper absolute as defined above, a ginger absolute as defined above and a tea resinoid as defined above;
a gentian absolute as defined above, a myrrh resinoid as defined above and an essential oil of mandarin petitgrain as defined above;
a gentian absolute as defined above, a myrrh resinoid as defined above and a ginger absolute as defined above;
a gentian absolute as defined above, a myrrh resinoid as defined above and a tea resinoid as defined above;
a gentian absolute as defined above, an essential oil of mandarin petitgrain as defined above and a ginger absolute as defined above;
a gentian absolute as defined above, an essential oil of mandarin petitgrain as defined above and a tea resinoid as defined above;
a gentian absolute as defined above, a ginger absolute as defined above and a tea resinoid as defined above;
a myrrh resinoid as defined above, an essential oil of mandarin petitgrain as defined above and a ginger absolute as defined above;
a myrrh resinoid as defined above, an essential oil of mandarin petitgrain as defined above and a tea resinoid as defined above;
a myrrh resinoid as defined above, a ginger absolute as defined above and a tea resinoid as defined above;
an essential oil of mandarin petitgrain as defined above, a ginger absolute as defined above and a tea resinoid as defined above;
a plant extract of rosemary as defined above, a black pepper absolute as defined above, a gentian absolute as defined above and a myrrh resinoid as defined above;
a plant extract of rosemary as defined above, a black pepper absolute as defined above, a gentian absolute as defined above and an essential oil of mandarin petitgrain as defined above;
a plant extract of rosemary as defined above, a black pepper absolute as defined above, a gentian absolute as defined above and a ginger absolute as defined above;
a plant extract of rosemary as defined above, a black pepper absolute as defined above, a gentian absolute as defined above and a tea resinoid as defined above;
a plant extract of rosemary as defined above, a black pepper absolute as defined above, a myrrh resinoid as defined above and an essential oil of mandarin petitgrain as defined above;
a plant extract of rosemary as defined above, a black pepper absolute as defined above, a myrrh resinoid as defined above and a ginger absolute as defined above;
a plant extract of rosemary as defined above, a black pepper absolute as defined above, a myrrh resinoid as defined above and a tea resinoid as defined above;
a plant extract of rosemary as defined above, a black pepper absolute as defined above, an essential oil of mandarin petitgrain as defined above and a ginger absolute as defined above;
a plant extract of rosemary as defined above, a black pepper absolute as defined above, an essential oil of mandarin petitgrain as defined above and a tea resinoid as defined above;
a plant extract of rosemary as defined above, a black pepper absolute as defined above, a ginger absolute as defined above and a tea resinoid as defined above;
a plant extract of rosemary as defined above, a gentian absolute as defined above, a myrrh resinoid as defined above and an essential oil of mandarin petitgrain as defined above;
a plant extract of rosemary as defined above, a gentian absolute as defined above, a myrrh resinoid as defined above and a ginger absolute as defined above;
a plant extract of rosemary as defined above, a gentian absolute as defined above, a myrrh resinoid as defined above and a tea resinoid as defined above;
a plant extract of rosemary as defined above, a gentian absolute as defined above, an essential oil of mandarin petitgrain as defined above and a ginger absolute as defined above;
a plant extract of rosemary as defined above, a gentian absolute as defined above, an essential oil of mandarin petitgrain as defined above and a tea resinoid as defined above;
a plant extract of rosemary as defined above, a gentian absolute as defined above, a ginger absolute as defined above and a tea resinoid as defined above;
a plant extract of rosemary as defined above, a myrrh resinoid as defined above, an essential oil of mandarin petitgrain as defined above and a ginger absolute as defined above;
a plant extract of rosemary as defined above, a myrrh resinoid as defined above, an essential oil of mandarin petitgrain as defined above and a tea resinoid as defined above;
a plant extract of rosemary as defined above, a myrrh resinoid as defined above, a ginger absolute as defined above and a tea resinoid as defined above;
a plant extract of rosemary as defined above, an essential oil of mandarin petitgrain as defined above, a ginger absolute as defined above and a tea resinoid as defined above;
a black pepper absolute as defined above, a gentian absolute as defined above, a myrrh resinoid as defined above and an essential oil of mandarin petitgrain as defined above;
a black pepper absolute as defined above, a gentian absolute as defined above, a myrrh resinoid as defined above and a ginger absolute as defined above;
a black pepper absolute as defined above, a gentian absolute as defined above, a myrrh resinoid as defined above and a tea resinoid as defined above;
a black pepper absolute as defined above, a gentian absolute as defined above, an essential oil of mandarin petitgrain as defined above and a ginger absolute as defined above;
a black pepper absolute as defined above, a gentian absolute as defined above, an essential oil of mandarin petitgrain as defined above and a tea resinoid as defined above;
a black pepper absolute as defined above, a gentian absolute as defined above, a ginger absolute as defined above and a tea resinoid as defined above;
a black pepper absolute as defined above, a myrrh resinoid as defined above, an essential oil of mandarin petitgrain as defined above and a ginger absolute as defined above;
a black pepper absolute as defined above, a myrrh resinoid as defined above, an essential oil of mandarin petitgrain as defined above and a tea resinoid as defined above;
a black pepper absolute as defined above, a myrrh resinoid as defined above, a ginger absolute as defined above and a tea resinoid as defined above;
a black pepper absolute as defined above, an essential oil of mandarin petitgrain as defined above, a ginger absolute as defined above and a tea resinoid as defined above;
a gentian absolute as defined above, a myrrh resinoid as defined above, an essential oil of mandarin petitgrain as defined above and a ginger absolute as defined above;
a gentian absolute as defined above, a myrrh resinoid as defined above, an essential oil of mandarin petitgrain as defined above and a tea resinoid as defined above;
a gentian absolute as defined above, a myrrh resinoid as defined above, a ginger absolute as defined above and a tea resinoid as defined above;
a gentian absolute as defined above, an essential oil of mandarin petitgrain as defined above, a ginger absolute as defined above and a tea resinoid as defined above;
a myrrh resinoid as defined above, an essential oil of mandarin petitgrain as defined above, a ginger absolute as defined above and a tea resinoid as defined above;
a plant extract of rosemary as defined above, a black pepper absolute as defined above, a gentian absolute as defined above, a myrrh resinoid as defined above and an essential oil of mandarin petitgrain as defined above;
a plant extract of rosemary as defined above, a black pepper absolute as defined above, a gentian absolute as defined above, a myrrh resinoid as defined above and a ginger absolute as defined above;
a plant extract of rosemary as defined above, a black pepper absolute as defined above, a gentian absolute as defined above, a myrrh resinoid as defined above and a tea resinoid as defined above;
a plant extract of rosemary as defined above, a black pepper absolute as defined above, a gentian absolute as defined above, an essential oil of mandarin petitgrain as defined above and a ginger absolute as defined above;
a plant extract of rosemary as defined above, a black pepper absolute as defined above, a gentian absolute as defined above, an essential oil of mandarin petitgrain as defined above and a tea resinoid as defined above;
a plant extract of rosemary as defined above, a black pepper absolute as defined above, a gentian absolute as defined above, a ginger absolute as defined above and a tea resinoid as defined above;
a plant extract of rosemary as defined above, a black pepper absolute as defined above, a myrrh resinoid as defined above, an essential oil of mandarin petitgrain as defined above and a ginger absolute as defined above;
a plant extract of rosemary as defined above, a black pepper absolute as defined above, a myrrh resinoid as defined above, an essential oil of mandarin petitgrain as defined above and a tea resinoid as defined above;
a plant extract of rosemary as defined above, a black pepper absolute as defined above, a myrrh resinoid as defined above, a ginger absolute as defined above and a tea resinoid as defined above;
a plant extract of rosemary as defined above, a black pepper absolute as defined above, an essential oil of mandarin petitgrain as defined above, a ginger absolute as defined above and a tea resinoid as defined above;
a plant extract of rosemary as defined above, a gentian absolute as defined above, a myrrh resinoid as defined above, an essential oil of mandarin petitgrain as defined above and a ginger absolute as defined above;
a plant extract of rosemary as defined above, a gentian absolute as defined above, a myrrh resinoid as defined above, an essential oil of mandarin petitgrain as defined above and a tea resinoid as defined above;
a plant extract of rosemary as defined above, a gentian absolute as defined above, a myrrh resinoid as defined above, a ginger absolute as defined above and a tea resinoid as defined above;
a plant extract of rosemary as defined above, a gentian absolute as defined above, an essential oil of mandarin petitgrain as defined above, a ginger absolute as defined above and a tea resinoid as defined above;
a plant extract of rosemary as defined above, a myrrh resinoid as defined above, an essential oil of mandarin petitgrain as defined above, a ginger absolute as defined above and a tea resinoid as defined above;
a black pepper absolute as defined above, a gentian absolute as defined above, a myrrh resinoid as defined above, an essential oil of mandarin petitgrain as defined above and a ginger absolute as defined above;
a black pepper absolute as defined above, a gentian absolute as defined above, a myrrh resinoid as defined above, an essential oil of mandarin petitgrain as defined above and a tea resinoid as defined above;
a black pepper absolute as defined above, a gentian absolute as defined above, a myrrh resinoid as defined above, a ginger absolute as defined above and a tea resinoid as defined above;
a black pepper absolute as defined above, a gentian absolute as defined above, an essential oil of mandarin petitgrain as defined above, a ginger absolute as defined above and a tea resinoid as defined above;
a black pepper absolute as defined above, a myrrh resinoid as defined above, an essential oil of mandarin petitgrain as defined above, a ginger absolute as defined above and a tea resinoid as defined above;
a gentian absolute as defined above, a myrrh resinoid as defined above, an essential oil of mandarin petitgrain as defined above, a ginger absolute as defined above and a tea resinoid as defined above;
a plant extract of rosemary as defined above, a black pepper absolute as defined above, a gentian absolute as defined above, a myrrh resinoid as defined above, an essential oil of mandarin petitgrain as defined above and a ginger absolute as defined above;
a plant extract of rosemary as defined above, a black pepper absolute as defined above, a gentian absolute as defined above, a myrrh resinoid as defined above, an essential oil of mandarin petitgrain as defined above and a tea resinoid as defined above;
a plant extract of rosemary as defined above, a black pepper absolute as defined above, a gentian absolute as defined above, a myrrh resinoid as defined above, a ginger absolute as defined above and a resinoid tea as defined above;
a plant extract of rosemary as defined above, a black pepper absolute as defined above, a gentian absolute as defined above, an essential oil of mandarin petitgrain as defined above, a ginger absolute as defined above and a tea resinoid as defined above;
a plant extract of rosemary as defined above, a black pepper absolute as defined above, a myrrh resinoid as defined above, an essential oil of mandarin petitgrain as defined above, a ginger absolute as defined above and a tea resinoid as defined above;
a plant extract of rosemary as defined above, a gentian absolute as defined above, a myrrh resinoid as defined above, an essential oil of mandarin petitgrain as defined above, a ginger absolute as defined above and a tea resinoid as defined above;
a black pepper absolute as defined above, a gentian absolute as defined above, a myrrh resinoid as defined above, an essential oil of mandarin petitgrain as defined above, a ginger absolute as defined above and a tea resinoid as defined above; or
a plant extract of rosemary as defined above, a black pepper absolute as defined above, a gentian absolute as defined above, a myrrh resinoid as defined above, an essential oil of mandarin petitgrain as defined above, a ginger absolute as defined above and a tea resinoid as defined above.

Preferably, the object of the present invention is a cosmetic composition as defined above comprising a plant extract of rosemary as defined above, a black pepper absolute as defined above and a gentian absolute as defined above.

More preferably, the object of the present invention is a cosmetic composition as defined above comprising a plant extract of rosemary as defined above, a black pepper absolute as defined above, a gentian absolute as defined above, a myrrh resinoid as defined above, an essential oil of mandarin petitgrain as defined above, a ginger absolute as defined above and/or a resinoid obtained from tea as defined above.

More preferably, the object of the present invention is a cosmetic composition as defined above comprising a plant extract of rosemary as defined above, a black pepper absolute as defined above, a gentian absolute as defined above and a myrrh resinoid as defined above.

More preferably, the object of the present invention is a cosmetic composition as defined above comprising a plant extract of rosemary as defined above, a black pepper absolute as defined above, a gentian absolute as defined above, a myrrh resinoid as defined above, an essential oil of mandarin petitgrain as defined above and a ginger absolute as defined above

Quite preferably, the object of the present invention is a cosmetic composition as defined above comprising a plant extract of rosemary as defined above, a black pepper absolute as defined above, a gentian absolute as defined above, a myrrh resinoid as defined above, an essential oil of mandarin petitgrain as defined above, a ginger absolute as defined above and a resinoid obtained from tea as defined above.

Finally, in another aspect, the present invention also relates to a method for protecting the skin from blue light comprising the selection of an area of skin on which said protection is sought, and the application to said area of at least one extract of aromatic raw material selected from a tea resinoid, a plant extract of rosemary, a black pepper absolute, a gentian absolute, an essential oil of mandarin petitgrain, a ginger absolute and a myrrh resinoid.

The present invention is illustrated without limitation by the following examples.

### EXAMPLE 1—COMPOSITIONS OF THE INVENTION

### Preparation

A composition comprising:
a resinoid obtained from leaves of Camelia sinensis—commercial reference R30632 (from Robertet);
a plant extract obtained from spent grains of Rosmarinus officinalis needles—commercial reference R30565 (from Robertet);
an absolute obtained from Piper nigra berries—commercial reference R21289 (from Robertet);
an absolute obtained from the roots of Gentiana lutea—commercial reference R20363 (from Robertet);
an absolute obtained from rhizomes of Zingiber officinale—commercial reference R20407 (from Robertet);
a resinoid obtained from Commiphora myrrha resin—commercial reference R10896 (from Robertet); and
an essential oil obtained by steam distillation of the leaves, branches and green fruits of Citrus reticulata—commercial reference R21500 (from Robertet).

To do this, the different ingredients are weighed and then mixed together. A neutral solvent (i.e. 96.2% ethyl alcohol) is added to facilitate the dissolution of the ingredients, some of which are more or less pasty and/or solid.

The composition reported in the following Table 1 is obtained.

TABLE 1
Composition of the invention

| Ingredient | % (p/p) |
|---|---|
| R30632 | 10% |
| R30565 | 19% |
| R21289 | 10% |
| R20363 | 1% |
| R20407 | 10% |
| R10896 | 10% |
| R21500 | 10% |
| Ethyl alcohol 96.2% | 30% |

### EXAMPLE 2—IN VIVO EFFICACY OF THE COMPOSITIONS OF THE INVENTION

### 2.1—Experimental Protocol

Primary human dermal fibroblasts, from a 57-year-old female donor, are thawed and amplified in flasks for a few days.

24 hours before the start of the test, each flask is treated as follows:
Flask n° 1: culture medium;
Flask n° 2: composition of example 1 at 30 ppm;
Flask n° 3: composition of example 1 at 20 ppm;
Flask n° 4: composition of example 1 at 10 ppm;
Flask n° 6: composition of example 1 at 2.5 ppm.

Day 1

The cells are incubated with the «Mitotracker Green» dye for 15 minutes.

Cells are washed with PBS, detached and seeded in a CYTOOplate with extra large Y microstructures at 3,000 cells/well in 10% serum.

1 h30 later, once the cells are attached and diffused on the microstructure, the medium is changed to medium containing less serum plus the active ingredient at 5 concentrations, incubated for 2 h at 37 degrees with 5% CO₂.

After 2 hours of treatment, the cells are irradiated with LEDs emitting at 447 nm for 1 hour corresponding to 20 mJ/cm2.

Hoechst is added for 15 min in each well to stain the nucleus

The medium is changed to wash off the nuclear stain and the cells are incubated in medium plus active ingredient.

Live imaging was performed on the LEICA microscope under a controlled environment at objective 40, with 49 fields/wells.

At the end of the imaging, the cells are fixed and the actin filaments are labeled with Phalloidin 555. The images were acquired on the Operetta HCS platform from Perkin Elmer.

### Blue Light Stress

A device developed by Genel is used to irradiate cells with 447 nm light. The device is made of LEDs and delivers a dose of 20 J/cm2.

Radiant light is the power of light received on a surface per unit area [W/cm2]=Dose [J/cm²]/time in seconds.

The radiant light emitted by a screen is about 18 to 40 μW/cm2 at a distance of 10 centimeters. The average screen usage time is 5 hours per day, i.e. 18,000 seconds per day, namely a daily dose emitted by a screen at 10 cm=light emitted*time=40 μW/cm2*18000 s/day=0.72 J/cm2/day.

By using the device at 20 J/cm² for 1 hour, it is simulated that the dose of 1 month (28 days) of screen at a distance of 10 cm.

### Mitochondrial Characterization by Image Analysis

A very large reading panel was developed with the aim of collecting as much information as possible about mitochondrial filament size and density to detect fragmentation and elongation phenotypes.

The mitochondrial network can be divided into several groups of filaments which are linked together: the basal unit is called «Tree». The number of Trees per array, as well as their total length (Average Total Tree Length) is an average across all single cells detected in each well.

Each Tree is divided into branches that define each other by either a junction or an endpoint. These branches are characterized by measuring their maximum size in the total network of a single cell.

### 2.2—Results

### Observations

Mitochondrial fluorescence images were acquired at the objective 40.

The mitochondrial network of untreated unstressed cells is nicely elongated/with nice extensions.

Cells treated with the composition of example 1 but not stressed by blue light show a well extended mitochondrial network similar to the control. (Negative control)

Blue light stress induces dramatic mitochondrial fragmentation. The number of «subunits» is increased by 3 and their length is reduced almost 4 times.

The same trend is detected on the measurement of ramifications with an average length decreased by 1.7 times.

Cells were treated in the flask with the composition of Example 1 for 24 h prior to plating as well as during blue light stress.

Treatment with the composition of Example 1 partially protects the cellular mitochondrial network from fragmentation by blue light.

### Statistics

Throughout the study, statistical tests were carried out according to:

1) An unpaired T-test followed by a Welch correction to compare:

The «untreated unstressed» condition with the «untreated stressful light» condition

2) An Anova to compare:
the «untreated, unstressed» condition with the «composition of example 1» condition;
the «untreated light stressed» condition with the «composition of example 1 in the ppm light stress» conditions;

with *p<0.05; **p<0.01; ***p<0.001; ****p<0.0001.

The results obtained are reported in Tables 2 to 9 below.

TABLE 2
NB Tree/Total (without stress)

| | | Doses of composition | | | | |
|---|---|---|---|---|---|---|
| | Untreated sample | according to example 1 (in ppm) | | | | |
| | (without stress) | 2.5 | 5 | 10 | 20 | 30 |
| | 0.135 | 0.140 | 0.088 | 0.093 | 0.108 | 0.114 |
| | 0.099 | 0.129 | 0.107 | 0.092 | 0.107 | 0.113 |
| | 0.119 | 0.095 | 0.108 | 0.103 | 0.141 | 0.134 |
| | 0.112 | 0.108 | 0.093 | 0.102 | 0.146 | 0.194 |
| | 0.142 | 0.132 | 0.131 | 0.108 | 0.162 | 0.158 |
| Average | 0.121 | 0.121 | 0.105 | 0.099 | 0.133 | 0.143 |

TABLE 3
NB Tree/Total (with stress)

| | | | Doses of composition | | | | |
|---|---|---|---|---|---|---|---|
| | Untreated sample | Untreated sample | according to example 1 (in ppm) | | | | |
| | (without stress) | (with stress) | 2.5 | 5 | 10 | 20 | 30 |
| | 0.135 | 0.436 | 0.326 | 0.290 | 0.335 | 0.407 | 0.217 |
| | 0.099 | 0.392 | 0.334 | 0.297 | 0.301 | 0.349 | 0.235 |
| | 0.119 | 0.311 | 0.274 | 0.225 | 0.227 | 0.227 | 0.195 |
| | 0.112 | 0.476 | 0.297 | 0.253 | 0.322 | 0.305 | 0.249 |
| | 0.142 | 0.409 | 0.310 | 0.287 | 0.309 | 0.342 | 0.368 |
| Average | 0.121 | 0.405 | 0.308 | 0.270 | 0.299 | 0.326 | 0.253 |
| % | — | — | 34.1 | 47.4 | 37.3 | 27.8 | 53.6 |
| protected | | | | | | | |

TABLE 4
Average Tree Length (without stress)

| | | Doses of composition | | | | |
|---|---|---|---|---|---|---|
| | Untreated sample | according to example 1 (in ppm) | | | | |
| | (without stress) | 2.5 | 5 | 10 | 20 | 30 |
| | 7.469 | 9.739 | 12.42 | 11.187 | 10.506 | 9.868 |
| | 10.905 | 10.234 | 9.808 | 10.653 | 9.594 | 9.138 |
| | 9.022 | 11.688 | 10.61 | 10.396 | 7.749 | 8.416 |
| | 9.347 | 10.204 | 11.62 | 11.911 | 6.918 | 5.972 |
| | 7.565 | 8.252 | 8.399 | 10.195 | 6.475 | 6.739 |
| Average | 8.862 | 10.023 | 10.571 | 10.868 | 8.248 | 8.027 |

TABLE 5
Average length of Tree (with stress)

| | | | Doses of composition according to | | | | |
|---|---|---|---|---|---|---|---|
| | Untreated sample | Untreated sample | example 1 (in ppm) | | | | |
| | (without stress) | (with stress) | 2.5 | 5 | 10 | 20 | 30 |
| | 7.469 | 2.166 | 3.14 | 3.437 | 2.996 | 2.556 | 4.977 |
| | 10.905 | 2.442 | 2.952 | 3.431 | 3.304 | 2.833 | 4.27 |
| | 9.022 | 3.313 | 3.496 | 4.486 | 4.087 | 4.325 | 5.255 |
| | 9.347 | 2.141 | 3.281 | 3.861 | 3.067 | 3.202 | 4.116 |
| | 7.565 | 2.222 | 3.32 | 3.36 | 3.255 | 2.834 | 2.8 |
| Average | 8.862 | 2.457 | 3.238 | 3.715 | 3.342 | 3.150 | 4.284 |
| % | — | — | 12.2 | 19.6 | 13.8 | 10.8 | 28.5 |
| protected | | | | | | | |

TABLE 6
NB branches/Total (without stress)

| | | Doses of composition | | | | |
|---|---|---|---|---|---|---|
| | Untreated sample | according to example 1 (in ppm) | | | | |
| | (without stress) | 2.5 | 5 | 10 | 20 | 30 |
| | 0.448 | 0.473 | 0.467 | 0.467 | 0.495 | 0.485 |
| | 0.428 | 0.44 | 0.433 | 0.48 | 0.458 | 0.469 |
| | 0.453 | 0.45 | 0.448 | 0.45 | 0.488 | 0.478 |
| | 0.439 | 0.441 | 0.461 | 0.461 | 0.482 | 0.555 |
| | 0.453 | 0.443 | 0.46 | 0.47 | 0.486 | 0.491 |
| Average | 0.444 | 0.449 | 0.454 | 0.466 | 0.482 | 0.496 |

TABLE 7
NB branches/Total (with stress)

| | | | Doses of composition according to | | | | |
|---|---|---|---|---|---|---|---|
| | Untreated sample | Untreated sample | example 1 (in ppm) | | | | |
| | (without stress) | (with stress) | 2.5 | 5 | 10 | 20 | 30 |
| | 0.448 | 0.764 | 0.642 | 0.585 | 0.673 | 0.742 | 0.59 |
| | 0.428 | 0.747 | 0.664 | 0.609 | 0.631 | 0.701 | 0.616 |
| | 0.453 | 0.677 | 0.59 | 0.531 | 0.566 | 0.609 | 0.568 |
| | 0.439 | 0.752 | 0.614 | 0.551 | 0.627 | 0.672 | 0.617 |
| | 0.453 | 0.749 | 0.578 | 0.58 | 0.586 | 0.672 | 0.673 |
| Average | 0.444 | 0.738 | 0.618 | 0.571 | 0.617 | 0.679 | 0.613 |
| % | — | — | 40.9 | 56.7 | 41.3 | 20.0 | 42.6 |
| protected | | | | | | | |

TABLE 8
Average branch length (without stress)

| | | Doses of composition | | | | |
|---|---|---|---|---|---|---|
| | Untreated sample | according to example 1 (in ppm) | | | | |
| | (without stress) | 2.5 | 5 | 10 | 20 | 30 |
| | 2.22 | 2.288 | 1.965 | 1.961 | 1.836 | 1.961 |
| | 2.135 | 2.232 | 2.192 | 1.848 | 2.002 | 1.949 |
| | 2.061 | 2.06 | 2.061 | 2.034 | 1.846 | 1.998 |
| | 2.087 | 2.214 | 2.028 | 1.933 | 1.972 | 1.688 |
| | 2.137 | 2.147 | 2.198 | 1.9 | 1.941 | 1.883 |
| Average | 2.128 | 2.188 | 2.089 | 1.935 | 1.919 | 1.896 |

TABLE 9
Average branch length (with stress)

| | | | Doses of composition according to | | | | |
|---|---|---|---|---|---|---|---|
| | Untreated sample | Untreated sample | example 1 (in ppm) | | | | |
| | (without stress) | (with stress) | 2.5 | 5 | 10 | 20 | 30 |
| | 2.22 | 1.157 | 1.44 | 1.65 | 1.333 | 1.151 | 1.575 |
| | 2.135 | 1.182 | 1.333 | 1.576 | 1.41 | 1.217 | 1.432 |
| | 2.061 | 1.348 | 1.586 | 1.767 | 1.639 | 1.487 | 1.526 |
| | 2.087 | 1.18 | 1.455 | 1.706 | 1.483 | 1.294 | 1.391 |
| | 2.137 | 1.156 | 1.642 | 1.639 | 1.545 | 1.258 | 1.412 |
| Average | 2.128 | 1.205 | 1.491 | 1.668 | 1.482 | 1.281 | 1.467 |
| % | | | 31.0 | 50.1 | 30.0 | 8.3 | 28.4 |
| protected | | | | | | | |

### Conclusion

Blue light stress induces strong fragmentation of the mitochondrial network in fibroblasts, increasing the number of mitochondrial subunits. Treatment with the composition of example 1 protects the cells from stress by blue light with a maximum effect at 5 ppm.

### EXAMPLE 3—IN VIVO EFFICACY OF THE COMPOSITIONS OF THE INVENTION

### 3.1—Experimental Protocol

At the end of the experiment detailed in Example 2, the plates are fixed in a formaldehyde solution and the cells are stained with Phalloidin. Images are acquired at *20 with the Operetta HCS instrument (Perkin Elmer) in three channels: microstructure, nucleus and F-actin.

A special analysis of the images was carried out with the aim of detecting single cells in the microstructure and measuring their area. The correctly plated cells counted have an area about 1800 μm².

### 3.2—Results

The obtained results are reported in Tables 10 to 13 below.

TABLE 10
Cell surface (without stress)

| | | Doses of composition | | | | |
|---|---|---|---|---|---|---|
| | Untreated sample | according to example 1 (in ppm) | | | | |
| | (without stress) | 2.5 | 5 | 10 | 20 | 30 |
| | 2062 | 2120 | 2204 | 2371 | 2242 | 2177 |
| | 2071 | 2071 | 2255 | 2417 | 2226 | 2292 |
| | 2019 | 2120 | 2244 | 2338 | 2429 | 2341 |
| | 2161 | 2155 | 2155 | 2256 | 2286 | 2119 |
| | 2007 | 2066 | 2140 | 2119 | 2147 | 2324 |
| Average | 2064 | 2106 | 2200 | 2300 | 2266 | 2250 |

TABLE 11
Cell surface (with stress)

| | | | Doses of composition according to | | | | |
|---|---|---|---|---|---|---|---|
| | Untreated sample | Untreated sample | example 1 (in ppm) | | | | |
| | (without stress) | (with stress) | 2.5 | 5 | 10 | 20 | 30 |
| | 2062 | 1779 | 2022 | 2083 | 2113 | 1978 | 2062 |
| | 2071 | 1906 | 2069 | 2130 | 2026 | 2016 | 2071 |
| | 2019 | 2033 | 2078 | 2226 | 2283 | 2231 | 2019 |
| | 2161 | 1913 | 2107 | 2122 | 2084 | 2009 | 2161 |
| | 2007 | 1759 | 1936 | 2076 | 1918 | 1821 | 2007 |
| Average | 2064 | 1878 | 2042 | 2127 | 2085 | 2011 | 2064 |
| % | — | — | 88.5 | 134.2 | 111.2 | 71.5 | 3.5 |
| protected | | | | | | | |

TABLE 12
% Cell Diffusion > 1800 (without stress)

| | | Doses of composition | | | | |
|---|---|---|---|---|---|---|
| | Untreated sample | according to example 1 (in ppm) | | | | |
| | (without stress) | 2.5 | 5 | 10 | 20 | 30 |
| | 63.64 | 72.90 | 81.69 | 90.14 | 81.10 | 80.86 |
| | 71.74 | 70.67 | 77.12 | 93.13 | 81.25 | 82.93 |
| | 64.03 | 71.43 | 80.81 | 84.91 | 91.94 | 87.13 |
| | 72.22 | 72.55 | 77.27 | 81.33 | 86.71 | 76.87 |
| | 66.46 | 71.35 | 70.39 | 77.54 | 75.28 | 76.70 |
| Average | 67.62 | 71.78 | 77.46 | 85.41 | 83.25 | 80.90 |

TABLE 13
% Cell Diffusion > 1800 (with stress)

| | | | Doses of composition according to | | | | |
|---|---|---|---|---|---|---|---|
| | Untreated sample | Untreated sample | example 1 (in ppm) | | | | |
| | (without stress) | (with stress) | 2.5 | 5 | 10 | 20 | 30 |
| | 63.64 | 41.73 | 66.43 | 71.43 | 71.59 | 67.38 | 57.75 |
| | 71.74 | 50.00 | 63.97 | 73.10 | 66.85 | 66.99 | 54.93 |
| | 64.03 | 58.23 | 70.70 | 83.12 | 88.36 | 82.04 | 68.06 |
| | 72.22 | 50.00 | 69.14 | 72.97 | 75.31 | 65.73 | 64.61 |
| | 66.46 | 39.41 | 58.38 | 69.94 | 66.44 | 55.21 | 43.52 |
| Average | 67.62 | 47.87 | 65.73 | 74.11 | 73.71 | 67.47 | 57.78 |
| % | — | — | 90.4 | 132.9 | 130.9 | 99.3 | 50.2 |
| protected | | | | | | | |

In the untreated condition, the cell surface is around 2063 μm² and 67% of the cells alone are nicely dispersed on the microstructure, with actin filaments on the corners.

The addition of the composition according to Example 1 without stress induces an increase in the cell surface as well as cell diffusion in a dose/response pathway of up to 10 ppm. The slight toxicity detected on the mitochondrial network has no impact on cell diffusion.

The stressful blue light negatively impacts cell diffusion capacity, reducing the number of diffused beautiful cells by 48%. The treatment with the composition according to Example 1 completely cancels the effects of blue light on cell diffusion at 5 and 10 ppm, with a viability percentage of 132% at 5 ppm.

### 3.3—Conclusion

The F-actin cytoskeleton is also impacted by stressful blue light, reducing the ability of cells to diffuse. The composition according to Example 1 also protects the fibroblasts from the stress induced by blue light on their diffusion capacities. Maximum effect was again observed at 5 ppm.

### EXAMPLE 4—COMPARATIVE EXAMPLE

We are trying to determine the absorption spectra of the following extracts:
a plant extract obtained from spent grains of Rosmarinus officinalis needles—commercial reference R30565 (from Robertet)—plant extract of rosemary;
an essential oil obtained from the branches and leaves of Rosmarinus officinalis—commercial reference R21614 (from Robertet)—rosemary essential oil;
an absolute obtained from Zingiber officinale rhizomes—commercial reference R20407 (from Robertet)—ginger absolute; and
an essential oil obtained from the roots of Zingiber officinale—commercial reference R20779 (at Robertet)—essential oil of ginger.

To do this, each sample is diluted to 1000 ppm in ethanol. Each dilution is then transferred to a well of a 96-well microplate. An absorption spectrum is then obtained using a TECAN Infinite 2000 pro spectrophotometer. The OD (Optical Densities) are measured in the wavelengths of the visible spectrum (400 nm-800 nm).

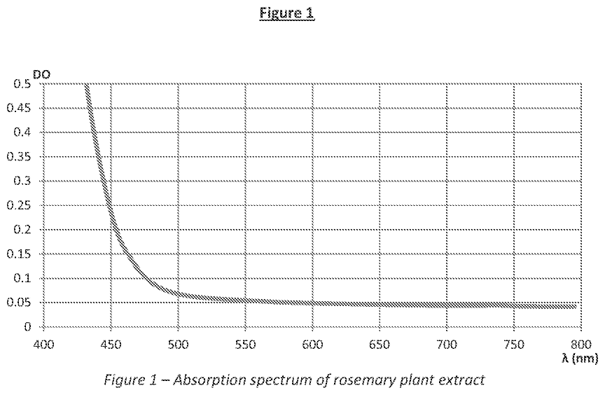

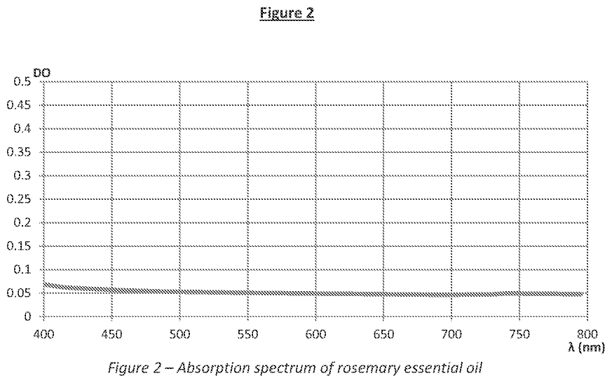

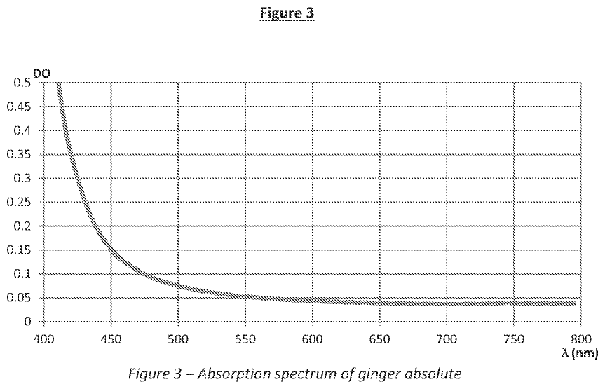

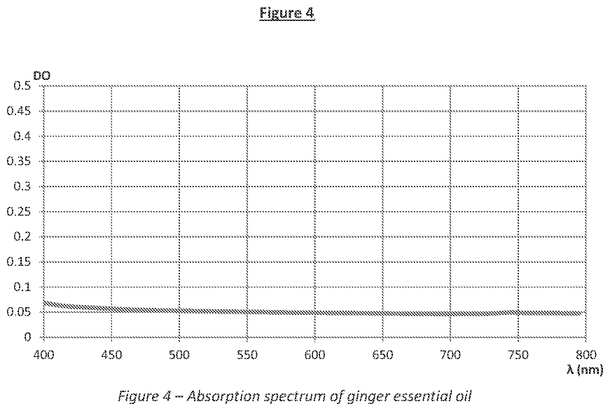

The absorption spectra obtained for plant extract of rosemary, rosemary essential oil, ginger absolute and ginger essential oil are respectively reported in FIGS. 1 to 4.

It appears that the plant extract of rosemary has an absorption between 400 and 500 nm, which confirms that this extract protects the skin against blue light. On the contrary, rosemary essential oil shows no absorption between 400 and 500 nm, and therefore cannot be used for protecting the skin from blue light.

Similarly, it appears that ginger absolute has an absorption between 400 and 500 nm, which confirms that this extract protects the skin against blue light. On the contrary, ginger essential oil shows no absorption between 400 and 500 nm, and therefore cannot be used for protecting the skin from blue light.

1. A method for protecting the skin from blue light comprising administering to a subject in need thereof at least one extract of aromatic raw material selected from the group consisting of a plant extract of rosemary containing at least 1% of rosmarinic acid, a black pepper absolute, a gentian absolute, a myrrh resinoid, an essential oil of mandarin petitgrain, a ginger absolute, and a tea resinoid.

2. The method according to claim 1, wherein the plant extract of rosemary contains at least 5% rosmarinic acid.

3. The method according to claim 1, wherein the plant extract of rosemary is obtained from spent grains of Rosmarinus officinalis needles.

4. The method according to claim 1, wherein the black pepper absolute is obtained from Piper nigra berries.

5. The method according to claim 1, wherein the gentian absolute is obtained from roots of Gentiana lutea.

6. The method according to claim 1, wherein the resinoid myrrh is obtained from Commiphora myrrha resin.

7. The method according to claim 1, wherein the essential oil of mandarin petitgrain is obtained by steam distillation of leaves of Citrus reticulata.

8. The method according to claim 1, wherein the ginger absolute is obtained from rhizomes of Zingiber officinale.

9. The method according to claim 1, wherein the ginger absolute contains more than 40% dry matter.

10. The method according to claim 1, wherein the tea resinoid is obtained from leaves of Camelia sinensis.

11. The method according to claim 1, wherein the at least one extract of aromatic raw material comprises the plant extract of rosemary, the black pepper absolute, and the gentian absolute.

12. The method according to claim 1, wherein the at least one extract of aromatic raw material comprises the plant extract of rosemary, the black pepper absolute, the gentian absolute, and the myrrh resinoid.

13. The method according to claim 1, wherein the at least one extract of aromatic raw material comprises the plant extract of rosemary, the black pepper absolute, the gentian absolute, the myrrh resinoid, the essential oil of mandarin petitgrain, and the ginger absolute.

14. The method according to claim 1, wherein the at least one extract of aromatic raw material comprises the plant extract of rosemary, the black pepper absolute, the gentian absolute, the myrrh resinoid, the essential oil of mandarin petitgrain, the ginger absolute, and the tea resinoid.

15. A cosmetic composition comprising at least three extracts selected from the group consisting of a plant extract of rosemary containing at least 1% of rosmarinic acid, a black pepper absolute, a gentian absolute, a myrrh resinoid, an essential oil of mandarin petitgrain, a ginger absolute, and a tea resinoid.

16. The cosmetic composition according to claim 15, wherein the at least three extracts comprise the plant extract of rosemary, the black pepper absolute, and the gentian absolute.

17. The cosmetic composition according to claim 16, wherein the at least three extracts further comprise the myrrh resinoid, the essential oil of mandarin petitgrain, the ginger absolute, and the tea resinoid.

18. The cosmetic composition according to claim 15, wherein the plant extract of rosemary is obtained from spent grains of Rosmarinus officinalis needles.

19. The cosmetic composition according to claim 15, wherein the black pepper absolute is obtained from Piper nigra berries.

20. The cosmetic composition according to claim 15, wherein the gentian absolute is obtained from roots of Gentiana lutea.

21. The cosmetic composition according to claim 15, wherein the resinoid myrrh is obtained from Commiphora myrrha resin.

22. The cosmetic composition according to claim 15, wherein the essential oil of mandarin petitgrain is obtained by steam distillation of leaves of Citrus reticulata.

23. The cosmetic composition according to claim 15, wherein the ginger absolute is obtained from rhizomes of Zingiber officinale.

24. The cosmetic composition according to claim 15, wherein the tea resinoid is obtained from leaves of Camelia sinensis.
